(19) United States
(12) Patent Application Publication
(10) Pub. No.: US 2006/0204472 A1
(43) Pub. Date: Sep. 14, 2006

(54) Multifunctional dendrimers and hyperbranched polymers as drug and gene delivery systems

(76) Inventors: Constantinos Paleos, Kallithea (GR); Dimitrios Tsiourvas, Athens (GR); Oreozili Sideratou, Pefki (GR)

Correspondence Address: MATHEWS, SHEPHERD, MCKAY, & BRUNEAU, P.A., 100 THANET CIRCLE, SUITE 306, PRINCETON, NJ, 08540, US

(21) Appl. No.: 10/545,307

(22) PCT Filed: Feb. 13, 2004
(86) PCT No.: PCT/GR04/00009

§ 371 Date: Mar. 31, 2006

(30) Foreign Application Priority Data
Feb. 13, 2003 (GR) 20030100069
May 2, 2003 (GR) 20030100194

(51) Int. Cl. A61K 48/00; A61K 31/785; C08G 63/48

(52) U.S. Cl. 424/78.27; 424/78.3; 525/54.2

(57) ABSTRACT

The present invention deals with the synthesis of multifunctional dendrimeric and hyperbranched polymers for application as drug delivery systems of bioactive pharmaceutical compounds and as gene delivery systems (carriers of genetic material), the latter through condensation with genetic material. Specifically, the present invention deals with the synthesis of multifunctional compounds based on appropriate dendrimeric or hyperbranched polymers at the terminal surface of which have been introduced functional groups X, Y, Z. In addition, for gene delivery to cells these multifunctional systems will become cationic for the formation of complexes with negatively charged genetic material. The functional groups render the delivery systems recognizable by complementary cell receptors. Furthermore they render the systems stable in the biological milieu and facilitate their transport through cell membranes.

### TECHNICAL FIELD

The present invention deals with the synthesis of multifunctional dendrimeric and hyperbranched polymers, particularly but not exclusively with the modification of their terminal surface groups in order that they can be used as efficient drug and gene delivery systems.

### PRIOR ART

The structural features of dendrimeric and hyperbranched polymers (dendritic polymers) and particularly the presence of nanocavities in their interior or also the presence of several groups at their external surface, render these polymers extremely useful candidates for drug and gene delivery applications. Bioactive pharmaceutical compounds can be encapsulated in the nanocavities while the surface groups can be appropriately modified allowing the preparation of multifunctional dendritic polymers. The application of dendrimers as drug carriers has been studied very recently and functional dendrimers have been prepared. These encapsulate bioactive pharmaceutical molecules in their nanocavities. This is due to the hydrophobic or, in certain other cases, to the hydrophilic environment, of the interior of the nanocavities which can encapsulate either lipophilic or hydrophilic compounds respectively. The structural features of the dendritic polymers, as these are described above, render possible the controlled release of the incorporated bioactive compound

It has been difficult to prepare multifunctional dendritic polymers which exhibit simultaneously all the desired properties so as to function effectively as drug carriers and specifically which exhibit biocompatibility and biodegradability, are biologically stable in order to circulate in the human body for prolonged periods of time, bear targeting ligands in order to be attached at cell-receptors and have the property of controlled release of the encapsulated bioactive compound. The absence of the one of the above properties renders a drug carrier ineffective. Consequently, several bioactive pharmaceutical compounds cannot be commercialized, if the drug carriers used do not exhibit multifunctional character as described above.

In gene therapy, viral vectors are extensively used as carriers of genetic material. Although viral vectors are in general effective, they have created problems to patients' health. For this purpose synthetic carriers e.g. non-viral vectors for genetic material have been recently introduced. Liposomes and dendrimers, for example have acquired significant interest for their application in gene therapy due to their safety as compared to viral carriers. Specifically, synthetic non-viral carriers for genetic material present insignificant risks of genetic recombinations in the genome. Transfection with synthetic, non-viral vectors is also characterized by low cell toxicity, high reproducibility and ease of application.

However, currently known synthetic vectors present disadvantages, due to their generally low effectiveness compared to viral vectors and to their inability for targeted gene expression. Specifically, for effective gene expression, genes must be transferred in the interior of cell nucleus and this procedure has to circumvent a series of endo- and exocell obstacles. These obstacles include: cell targeting, effective transport of the carriers together with genetic material they carry through cell membranes and the need for the carriers' release from the endosome following endocytosis.

For the synthetic carriers that have been described in the literature, some or all of these difficulties have been addressed, without however achieving the desired final objective. The present invention aims to simultaneously solve or address all of the abovementioned problems by the introduction of appropriate functional groups at the surface of the dendrimers or hyperbranched polymers. The above-mentioned difficulties require the development of novel and effective carriers that will transport the genetic material to the cell nucleus. Specifically, these carriers should simultaneously have the ability of targeting, exhibit stability in biological systems, have the ability of effective transport together with the attached genetic material through cell membranes and the possibility of the latter complex to be released from the endosome following endocytosis.

Such stable and effective synthetic gene carriers can be dendrimers or hyperbranched polymers. Dendrimers and hyperbranched polymers may be provided as stable nano-particles in contrast to liposomes that are usually unstable. The size of the dendrimers depend on their generation while the diversity of functional groups that can conveniently be introduced at their surface affect crucially their properties and consequently their applications.

### SUMMARY OF THE INVENTION

An objective of the present invention is to prepare multifunctional dendritic polymers which may be used as effective drug carriers for bioactive pharmaceutical compounds and genetic material. Preferred dendritic polymers include symmetric dendrimeric polymers and non-symmetrical hyperbranched polymers. By the application of these multifunctional dendrimers and hyperbranched polymers (dendrimeric polymers), it may be possible that pharmaceutical compounds can be commercialized, which otherwise would not be possible with conventional carriers. In addition, genes can be transfected to cells for gene therapy.

Hyperbranched polymers have not been extensively described as drug carriers. Their application is of significant interest because of their facile preparation and low price compared to dendrimeric polymers.

The terminal groups of the dendrimeric and hyperbranched polymers can be appropriately modified so as to become multifunctional, and permit pharmaceutical compounds to be encapsulated in their nanocavities.

Appropriately selected structural features of dendrimeric and hyperbranched polymers render these molecules simultaneously: biocompatible and biodegradable. Also, appropriate targeting ligands may be carried so as to be attached to cell-receptors, and the molecules may exhibit biological stability in order to circulate for prolonged periods of time in biological fluids. Controlled release of the encapsulated pharmaceutical compound may be permitted.

When these polymers are positively charged on their surface they can form complexes upon interaction with oligonucleosides or DNA.

The present invention reveals the preparation of multifunctional dendritic polymers, which in addition to their positively charged surface that leads to the formation of complexes with the negative charged DNA, they also bear functional groups, as those are described below, which facilitate the transport of genetic material.

The characteristic structural features of the proposed polymers that render these polymers useful, among others, for biomedical applications are the following:
a. The presence of functional groups at the surface of dendrimeric or hyperbranched polymers. These can be introduced in stages,
b. The presence of nanocavities in the interior of the polymers in which it is possible to encapsulate various chemical compounds, depending on their nano-environment. This latter property of these compounds finds particular application in their use as drug carriers.
c. When used for gene delivery the presence of cationic charges in these polymers is required since they will interact with the negatively charged DNA leading to the formation the respective complexes. The so-formed complexes may be introduced through endocytosis in the nucleus for gene therapy.

According to the present invention, there is provided dendrimeric polymers with symmetric chemical structure and non-symmetric hyperbranched polymers, characterized in that they are modified so as to exhibit:
at least one atom of a chemical element able to form three or more chemical bonds,
various different terminal functional groups bonded to said at least one atom, which terminal functional groups collectively a) have low toxicity or no toxicity at all, b) render the molecules of the above polymers recognizable from the complementary receptors of the cells, c) render the polymers stable in the organism's biological environment and d) facilitate the transport of the said polymers through cell membranes.

Preferably, the polymers are cationized for the formation of complexes with DNA when the said compounds are destined to be gene delivery systems, e.g. carriers of genetic material.

Conveniently, the polymers may be cationized by introducing ammonium, quatemary ammonium or guanidinium groups at the terminal groups of the dendrimer.

Advantageously, the atom of a chemical element is able to form three or more chemical bonds, may be nitrogen or other appropriate characteristic group, e.g. carbon or silicon.

Preferably, the modified dendrimeric polymer may be the diaminobutane poly(propylene imino) dendrimer (DAB), or other dendrimeric molecules of similar structure, e.g. PAMAM dendrimers.

Conveniently, the modified hyperbranched non-symmetric polymers may be derived from the poly-condensation of an anhydride e.g. succinic, phthallic or tetrahydrophthalic anhydride with a dialkyl amine e.g. diisopropylamine.

Advantageously, the modified hyperbranched non-symmetric polymers may be derived from the anionic polymerization of epoxide derivatives with 1,1,1 tri(hydroxyalkyl) propane.

Conveniently, the modified hyperbranched non-symmetric polymers may be derived from the anionic polymerization of glycidol with 1,1,1 tri(hydroxymethyl)propane (PG-5).

Conveniently, the modified dendrimeric polymer or modified hyperbranched non-symmetric polymer may have at their surface functional groups that include polymeric chains of diversified molecular weight, e.g. polyalkylene glycol and preferably poly(ethyleneglycol).

Advantageously, the modified dendrimeric polymer or modified hyperbranched non-symmetric polymer may comprise functional groups that include at least one group that is complementary to a receptor site of a cell, e.g. a guanidinium group, a carbohydrate (e.g. mannose, glycose, galactose), a folate, an RGD receptor, a nucleobase moiety (such as adenine, thymine, guanine, cytosine) or a barbiturate.

Advantageously, the modified dendrimeric polymer or modified hyperbranched non-symmetric polymer may comprise functional groups that include at least one group that facilitates the transport of the dendrimeric polymer or modified hyperbranched polymer together with any encapsulated active drug ingredient or genetic material through a cell membrane, e.g. a guanidinium moiety, an oligoarginine or polyarginine derivative or a polypropylene oxide moiety.

Conveniently, the modified dendrimeric polymer or modified hyperbranched non-symmetric polymer may comprise functional groups that include at least one targeting ligand, e.g. a guanidinium group, a carbohydrate (e.g. mannose, glycose, galactose), a folate, an RGD receptor, a nucleobase moiety (such as adenine, thymine, guanine, cytosine) or a barbiturate.

Preferably, the modified dendrimeric polymers and modified hyperbranched non-symmetric polymers may be used as drug carriers of bio-active pharmaceutical compounds, or for carrying genetic material.

Conveniently, the bio-active pharmaceutical compound carried by the modified dendrimeric polymers or modified hyperbranched non-symmetric polymers may be betamethasone or betamethasone derivatives.

The present invention also provides a method for the synthesis of multi-functional dendrimers and hyperbranched polymers in order that they can be used as drug carriers of bioactive pharmaceutical compounds, which method is characterized in that the surface of these polymers is modified in stages that comprise:
a. Substitution of the amino groups or other toxic groups of the surface, with hydroxy, carboxylic or quaternary ammonium groups, or other non-toxic groups.
b. Introduction of polymeric chains of diversified molecular weight at the surface of the dendrimeric carriers or of the hyperbranched polymers, as for instance of poly(ethyleneglycol) (PEGylation) so that the polymers are thus protected from the MPS (Mononuclear Phagocyte System) of the organism.
c. Introduction of recognizable groups complementary to the receptors or to the tissues i.e. of the guanidinium group, carbohydrate moieties (mannose, glycose, galactose), folate or RGD receptor, nucleobase moieties (adenine-thymine, guanine-cytosine) or barbiturate group, so as to enhance the targeting ability of the carrier.
d. Introduction of groups that facilitate the transport of the carriers together with the encapsulated active drug ingredient through cell membranes, such as guanidinium moieties, oligo-arginine or poly-arginine derivatives or polypropylene oxide moieties.

Preferably, the method comprises:

the initial reaction of external amino or hydroxy groups of dendrimers or hyperbranched polymers is performed with appropriate protective polymers, bearing reactive groups at one end such as isocyanate, epoxide or N-hydroxysuccinimide,

subsequent reaction of the greatest portion of amino groups of the obtained polymer is performed with ethylisocyanate for the replacement of toxic amino groups,

subsequent reaction of the previously obtained polymer for the transformation of amino groups to recognizable groups as for example guanidinium groups,

subsequent introduction of a group or groups which facilitate the transport of the carriers through cell membranes as for instance polyarginine or propyleneoxide chains.

Conveniently, the said polymers are cationized for the formation of complexes with DNA when the said compounds are destined to be gene delivery systems, e.g. they are destined to be carriers of genetic material.

Advantageously, the method is characterized in that when the toxic group of the surface is an amino group, a small aliphatic chain having less than eight carbon atoms, preferably two or three carbon atoms may be introduced for its replacements.

The present invention provides a pharmaceutical formulation which comprises bio-active pharmaceutical compound or genetic material encapsulated in a modified multifunctional dendrimeric or modified multifunctional hyperbranched non-symmetric polymer.

The present invention also provides a method for producing a pharmaceutical formulation for delivering a bio-active pharmaceutical compound or genetic material, which method comprises

synthesizing a symmetric dendrimer or a non-symmetrical hyperbranched polymer by modifying the surface of this polymer in stages that comprise: a. Substitution of the amino groups or other toxic groups of the surface, with hydroxy, carboxylic or quaternary ammonium groups, or other non-toxic groups. b. Introduction of polymeric chains of diversified molecular weight at the surface of the dendrimeric carriers or of the hyperbranched polymers, as for instance of poly(ethyleneglycol) (PEGylation) so that the polymers are thus protected from the MPS (Mononuclear Phagocyte System) of the organism. c. Introduction of recognizable groups complementary to the receptors or to the tissues i.e. of the guanidinium group, carbohydrate moieties (mannose, glycose, galactose), folate or RGD receptor, nucleobase moieties (adenine-thymine, guanine-cytosine) or barbiturate group, so as to enhance the targeting ability of the carrier. d. Introduction of groups that facilitate the transport of the carriers together with the encapsulated bio-active pharmaceutical compound through cell membranes, such as guanidinium moieties, oligo-arginine or poly-arginine derivatives or polypropylene oxide moieties; and

encapsulating the bio-active pharmaceutical compound or genetic material with the said modified polymer.

Preferably, the said polymers are cationized for the formation of complexes with DNA when the said compounds are destined to be carriers of genetic material. Conveniently, the modified dendrimeric polymer or modified hyperbranched non-symmetric polymer that include an encapsulated bio-active pharmaceutical compound or that carries genetic material is for use in therapy.

Advantageously, the modified dendrimeric polymer or modified hyperbranched non-symmetric polymer that include an encapsulated bio-active pharmaceutical compound or that carry genetic material in therapy is for use for manufacture of a pharmaceutical dosage form.

Conveniently, the modified dendrimeric polymer or modified hyperbranched non-symmetric polymer that include an encapsulated bio-active pharmaceutical compound or that carry genetic material is for use in the manufacture of a medicament for treating the same disease or condition as the compound or the genetic material.

### DESCRIPTION OF THE INVENTION

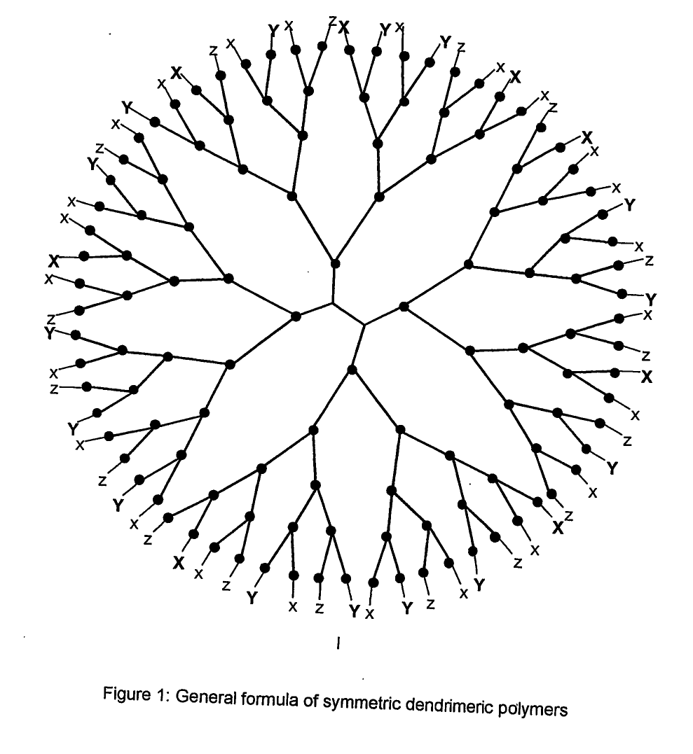

In one embodiment the present invention relates to the synthesis of multifunctional symmetric dendrimers. These are illustrated by the general formula (I) shown in FIG. 1. Such polymers may be, for example, diaminobutane poly(propylene imino) dendrimers.

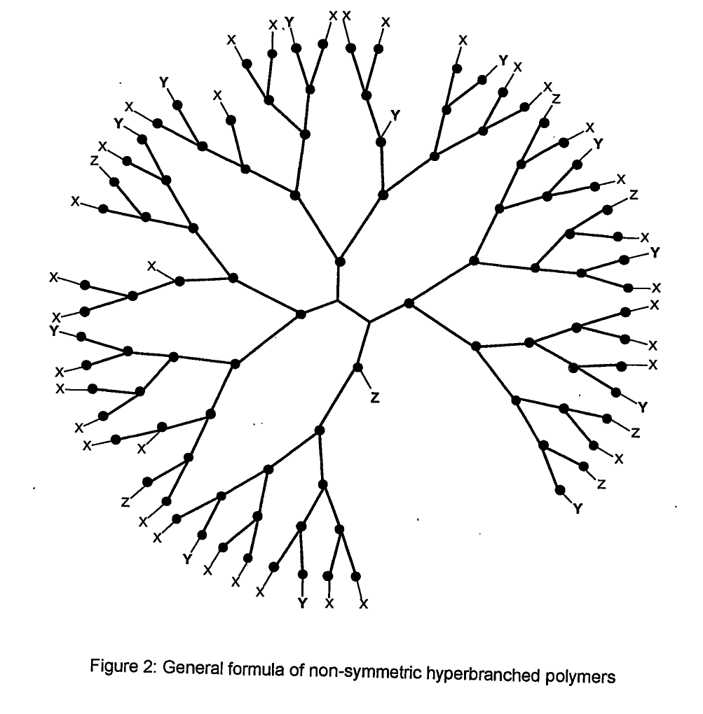

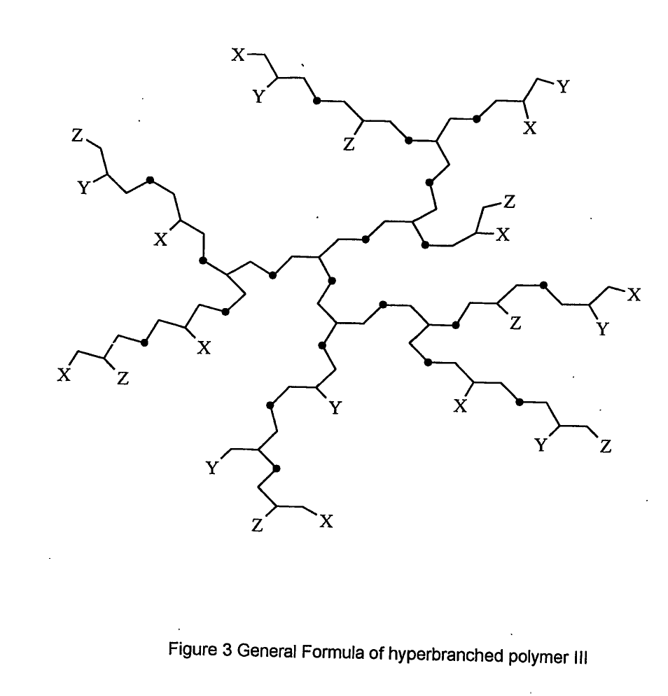

The present invention also relates to the synthesis of multifunctional non-symmetric hyperbranched polymers. These are illustrated by the general formula (II) shown in FIG. 2 and hyperbranched polymers of formula (Ill) shown in FIG. 3. Such non-symmetric polymers are, for example, the polymers resulting from the poly-condensation of succinic, phthalic or tetrahydrophthalic anhydride with diisopropylamine or from the anionic polymerization of glycidol with 1,1,1 tri(hydroxymethyl)propane.

In the formulas I, II and IlI the symbol (●) is an atom of a chemical element which can form three or more chemical bonds, for instance nitrogen or other appropriate characteristic group, for instance tertiary amino group, the straight line (-) corresponds to an aliphatic chain and the external functional groups X, Y, Z can collectively: a) render the molecules of the above polymers recognizable from the complementary receptors of the cells, b) render the above polymers stable in biological environment and c) facilitate the transport of these polymers through cell membranes.

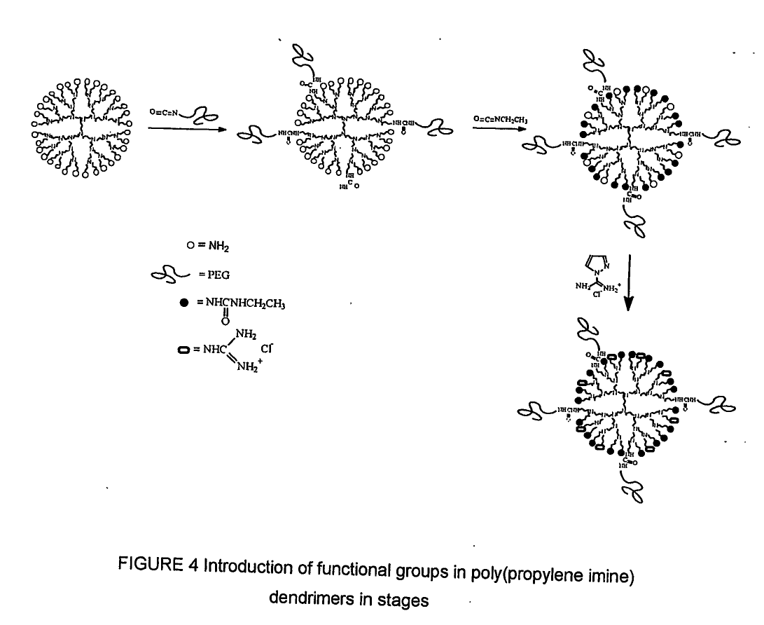

The characteristic structural features for the polymers described in the present invention, which make them useful, among others, for biomedical applications are the following: a) the presence of functional characteristic groups at the surface of the dendrimers or hyperbranched polymers, which result from their stepwise introduction at the surface of the polymers as for example shown in FIG. 4 and b) the presence of nanocavities in the interior of polymers in which it is possible that a variety of chemical compounds be encapsulated, depending on their nano-environment.

The modification of the surface of the dendrimers or hyperbranched polymers (molecular engineering of dendrimeric or hyperbranched polymers' surfaces) with the introduction at a first stage of positive charges, is capable to render the polymers appropriate for the binding of negatively charged genetic material (DNA, plasmids, oligonucleosides). The so-formed complexes of dendrimeric or hyperbranched polymeric carriers-genetic material are finally introduced through endocytosis in the nucleus for gene therapy.

For the preparation of such multi-functional dendrimeric and hyperbranched polymers, which are the objects of the present invention, commercially available dendrimers were used, purchased, for instance, from the company DSM and sold under the names DAB-32 and DAB-64. In appropriate reactors and under proper experimental conditions their structure was modified by a step-wise introduction of functional groups. In FIG. 4 is shown a scheme of reactions for the synthesis, for instance, of a multifunctional dendrimeric drug delivery system.

In another embodiment of the invention, instead of DAB, PAMAM dendrimers may equally be employed in appropriate reactors.

In the present invention a bioactive compound may be primarily introduced in the interior of the nanocavities of the dendrimers or of the hyperbranched polymers while on their external surface appropriate functional groups were introduced aiming at the formation of nano-sized carriers, which collectively have the following characteristics: they have low or no toxicity, they are stable in the biological milieu and they possess targeting and transport ability to specific cells.

When using dendrimers or hyperbranched polymers as appropriate carriers of genetic material (for gene delivery), positive charges are introduced for binding the negatively charged genetic material (DNA, plasmids, oligonucleosides), e.g. by introducing ammonium, quaternary ammonium or guanidinium ions at the terminal groups of the dendrimer or the hyperbranched polymer, as discussed below.

Subsequently, various functional groups are introduced at the surface of the dendrimers or of the hyperbranched polymers with final objective the transport of genetic material in the nucleus of the cells. Specifically, non-toxic dendrimers or hyperbranched polymers are selected, or alternatively the starting compounds are modified so as to be rendered non-toxic and biocompatible.

Subsequently, functional groups are introduced which: i) render the complexes of DNA-carriers stable in biological environment, ii) provide the property of targeting specific cells or tissues, iii) facilitate their transport through membranes and iv) have the ability of being released from the endosome following endocytosis.

The so-formed complexes of dendrimers or hyperbranched polymers with genetic material may be finally introduced through endocytosis to the cell. The genetic material finally enters the nucleus for gene therapy through an intracellular process.

All these properties are achieved with the processes mentioned below according to which the external terminal groups of the dendrimers or of the hyperbranched polymers are properly modified (molecular engineering of dendrimeric or hyperbranched polymers surfaces following established synthetic organic chemistry processes in an appropriate series of reactions) in order to achieve:
a) Substitution of the toxic terminal groups; for instance the amino groups, with non-toxic, e.g. with a hydroxy, carboxylic or quaternary ammonium group
b) Introduction of polymeric chains of diversified molecular weight at the surface of the dendrimeric carriers or of the hyperbranched polymers, as for instance of poly(ethyleneglycol) (PEGylation). The polymers are thus protected from the MPS (Mononuclear Phagocyte System) of the organism
c) Introduction of recognizable groups, complementary to the receptors of the cells e.g. of the guanidinium group, carbohydrate moieties (mannose, glycose, galactose), folate or RGD receptor, nucleobase moieties (adenine, thymine, guanine, cytosine) or of the barbiturate group, in order to enhance the targeting ability of the carrier
d) Introduction of groups that facilitate the transport of the carriers together with the encapsulated active drug ingredient or gene through cell membranes, such as guanidinium moieties, oligoarginine or polyarginine derivatives or polypropylene oxide moieties. Positively charged moieties such as ammonium, quatemary ammonium, guanidinium may be introduced for the formation of complexes with genetic material (DNA, plasmids, oligonucleosides).

The synthesis of such multifunctional dendrimers may be achieved by employing commercially available dendrimers or hyperbranched polymers. An indicative example, showing the steps for the synthesis of a multifunctional dendrimer is shown in FIG. 4.

Initially the external amino or hydroxy groups of the dendrimers or hyperbranched polymers may be reacted with selected molecular weight poly(ethyleneglycol) polymers which bear reactive groups, for example isocyanate, epoxide or N-hydroxysuccinimide moieties. Following this first stage, the majority of the remaining amino groups of the dendrimer obtained were reacted, for example with ethyl isocyanate, to reduce the presence of the toxic primary amino group at the external surface. In a third stage, the last remaining primary amino groups may be transformed to targeting groups, for instance guanidinium groups. In another stage, groups may be introduced that facilitate the transport of drug carriers together with the encapsulated active ingredient through cell membranes, for instance oligoarginine or polyarginine moieties. In the present case a guanidinium group, introduced as a targeting ligand can facilitate the transport through cell membranes of the delivery system encapsulating the active drug ingredient. Cationization of the dendrimers or hyperbranched polymers was required for the attachment of the negatively charged genetic material to the dendritic polymer for the formation of the respective stable complex with the genetic material which will be transfected to the cell.

The above mentioned reactions can take place in aqueous medium at room temperature. The purification of products was performed by passage of the by-products through a semi-permeable membrane by dialysis.

Typical dendrimers or hyperbranched polymers that may be used in the present invention, are for example, the symmetric diaminobutane poly(propylene imino) dendrimers or non-symmetric hyperbranched polymers, for example polymers resulting from the poly-condensation of succinic, phthalic or tetrahydrophthalic anhydride with diisopropylamine or from anionic polymerization of glycidol with 1,1,1 tri(hydroxymethyl)propane.

The polymers which can be used as a protective coating for dendrimers are, for example, polyethylene glycol with varying molecular weight that bears active groups for reacting with dendrimers or hyperbranched polymers, as for instance, isocyanate, epoxide or N-hydroxysuccinimide moieties, for example the isocyanate derivative of methoxypoly(ethyleneglycol) of average molecular weight 5,000 was used.

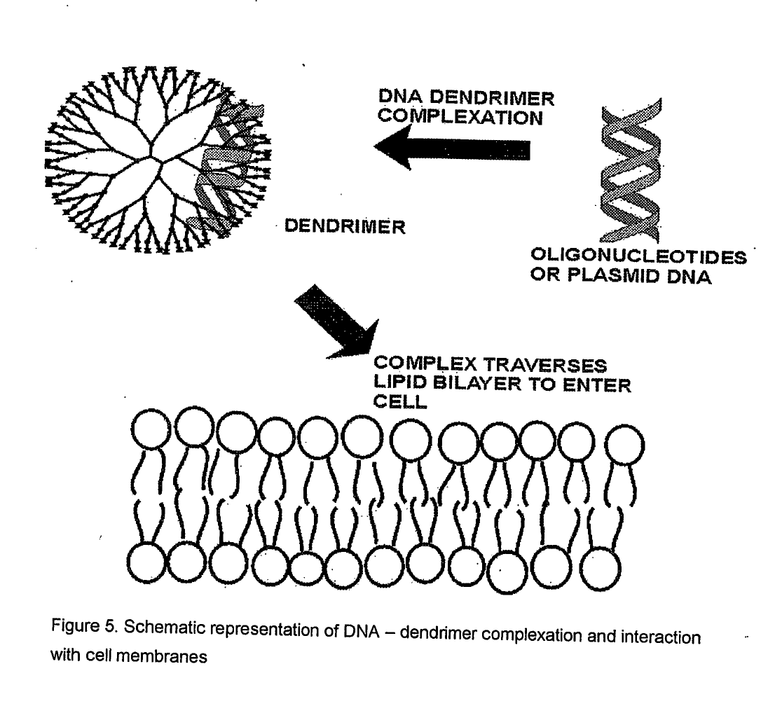

The substitution or reaction of toxic groups, as for instance of amino group, can be achieved by reaction with alkylisocyanates or alkylepoxides. The latter transform the primary amino group to secondary aminoalcohols. In the present invention ethylisocyanate is preferred, since it conveniently reacts with the primary amino group. Also, for the introduction of the targeting ligand, which in the example mentioned above is the guanidinium group, 1H-pyrazolo-1-carboxamidine hydrochloride may be used for the transformation of the external primary amino group of the dendrimer in question to this group. The guanidinium group as well as oligo- and polyarginine moieties facilitate the transport of the carrier through cell membranes. For gene delivery applications the preparation of the complex and its transport is shown schematically in FIG. 5.

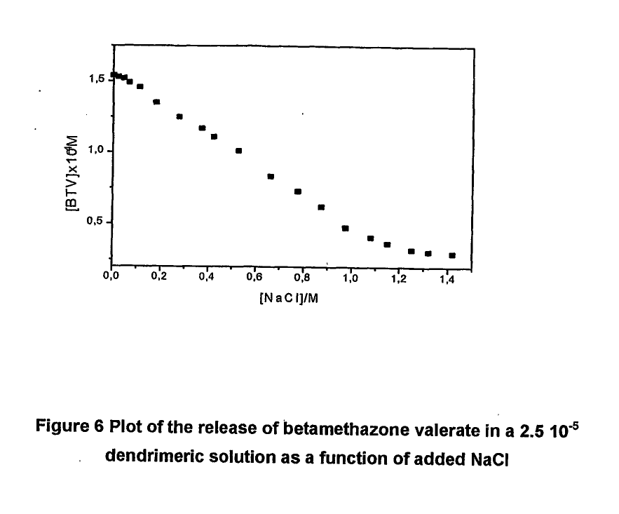

Examples of the use of dendrimers as drug carriers were performed employing lipophilic bioactive compounds, which are completely insoluble in water, like corticosteroids, as for example, betamethasone valerate. It was found that these compounds are solubilized in the interior of multifunctional dendrimers up to 14.5%. They are protected from poly(ethyleneglycol) chains (PEG) and they have the guanidinium groups as targeting ligands, which render the polymer capable of targeting cell or tissue receptors. It has also been established that betamethasone valerate remains encapsulated in these multifunctional dendrimers even in acidic environment. However, with the addition of aqueous NaCl solution the bioactive corticosteroid compound is released from the nanocavities of the dendrimers (FIG. 6).

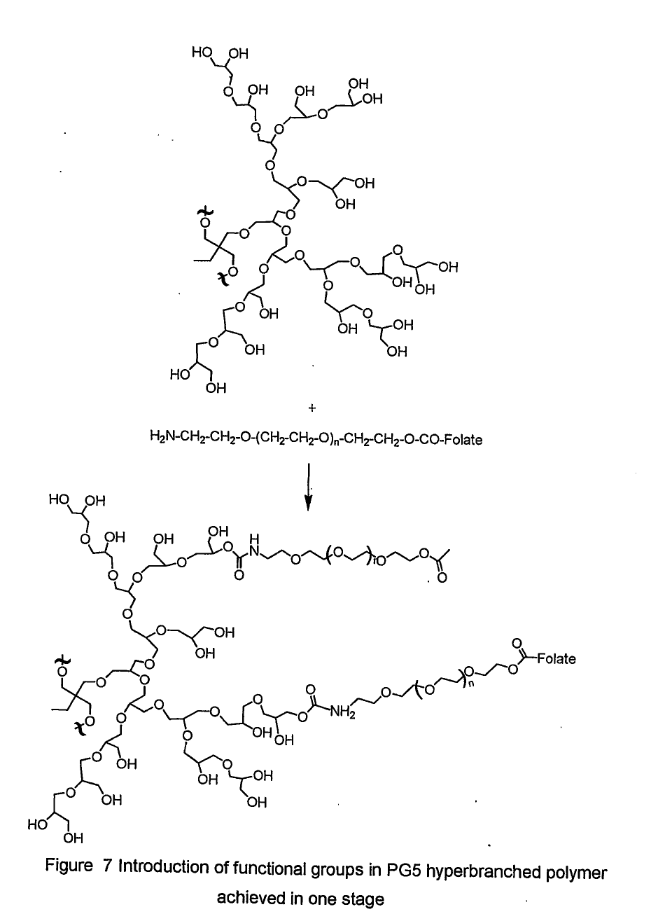

Due to the common structural features of the dendrimeric polymers with the similarly multifunctional hyperbranched polymers it is strongly anticipated that the latter polymers will show similar or almost the same behaviour and properties as drug carriers to those originating from multifunctional dendrimers. The reaction Scheme for the synthesis of multifunctional hyperbranched polymers based on a commercially available polymer, e.g. PG-s5 is shown in FIG. 7.

In this specification quantities mentioned in the examples below are in moles unless indicated otherwise.

### EXAMPLES

Materials and Methods

Diaminobutane poly(propylene imine) dendrimer of the 4^{th} and 5^{th} generation with 32 and 64 amino groups respectively at the external surface, (shown with No. 1 in the Scheme below—DAB-32 and DAB64, DSM Fine Chemicals) were used as starting dendrimeric polymers.

Methoxypoly(ethylene glycol)-isocyanate, (shown with No. 2 in the Scheme below—MW 5000, Shearwater Polymers, INC), ethylisocyanate (Aldrich) and 1H-pyrazolo-1-carboxamidine hydrochloride (Fluka), (shown with No. 3 in the Scheme below), were used for dendritic polymers multifunctionalization.

Betamethasone valerate, (shown with No. 4 in the Scheme below) which is a lipophilic drug, was provided by EFFECHEM S.R.L., Italy and it was used in encapsulation and release studies.

Glycidyltrimethylammonium chloride, (shown with No. 5 in the Scheme below), and Folic acid, (shown with No. 6 in the Scheme below), were purchased from Fluka. Hyperbranched polyether polyol, (shown with No. 7 in the Scheme below—MW 5000, PG-5) were purchased from Hyperpolymers GmbH and used after lyophilization.

The above mentioned dendritic polymers and basic organic starting chemicals are shown in the Scheme below. 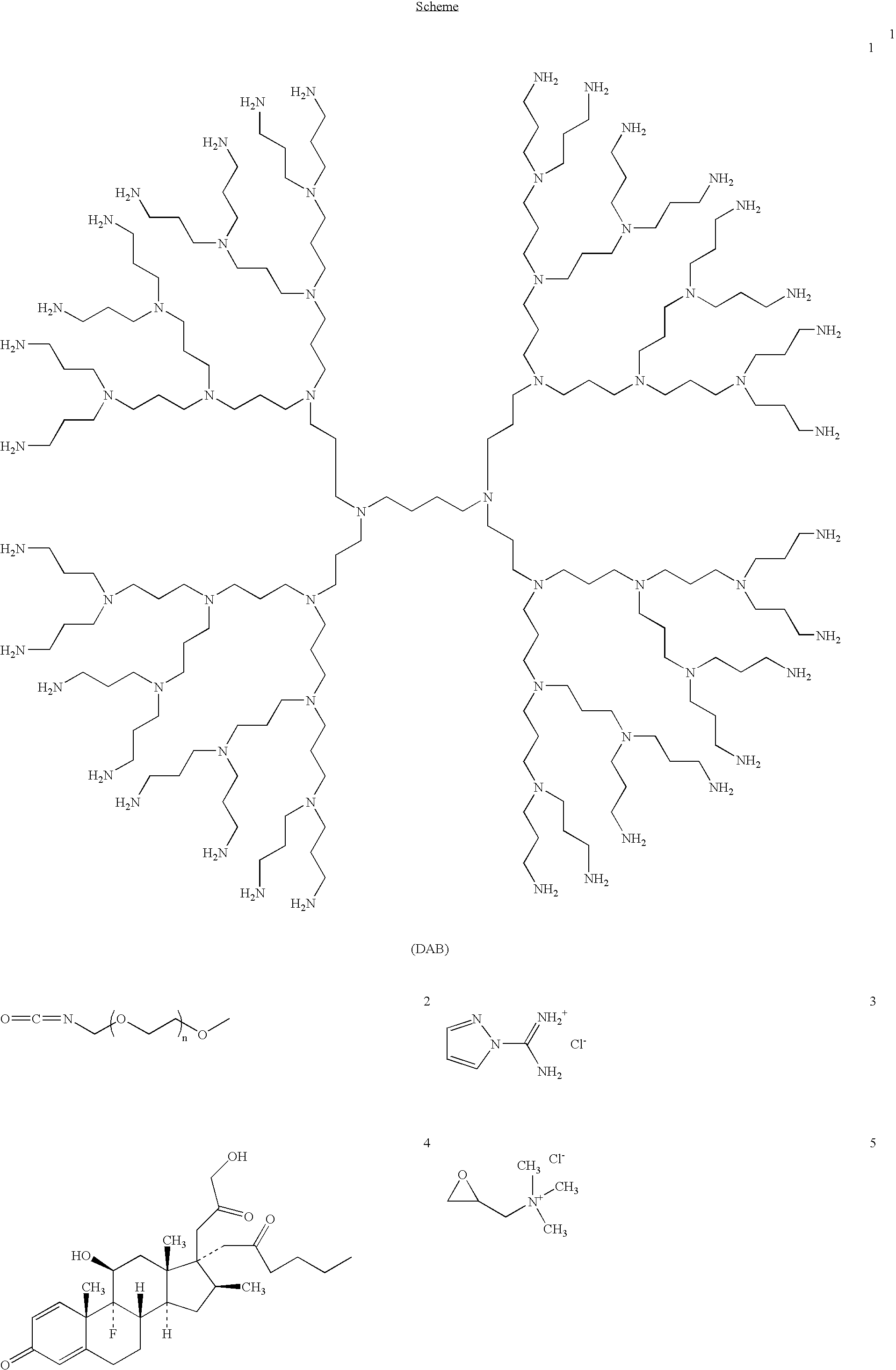 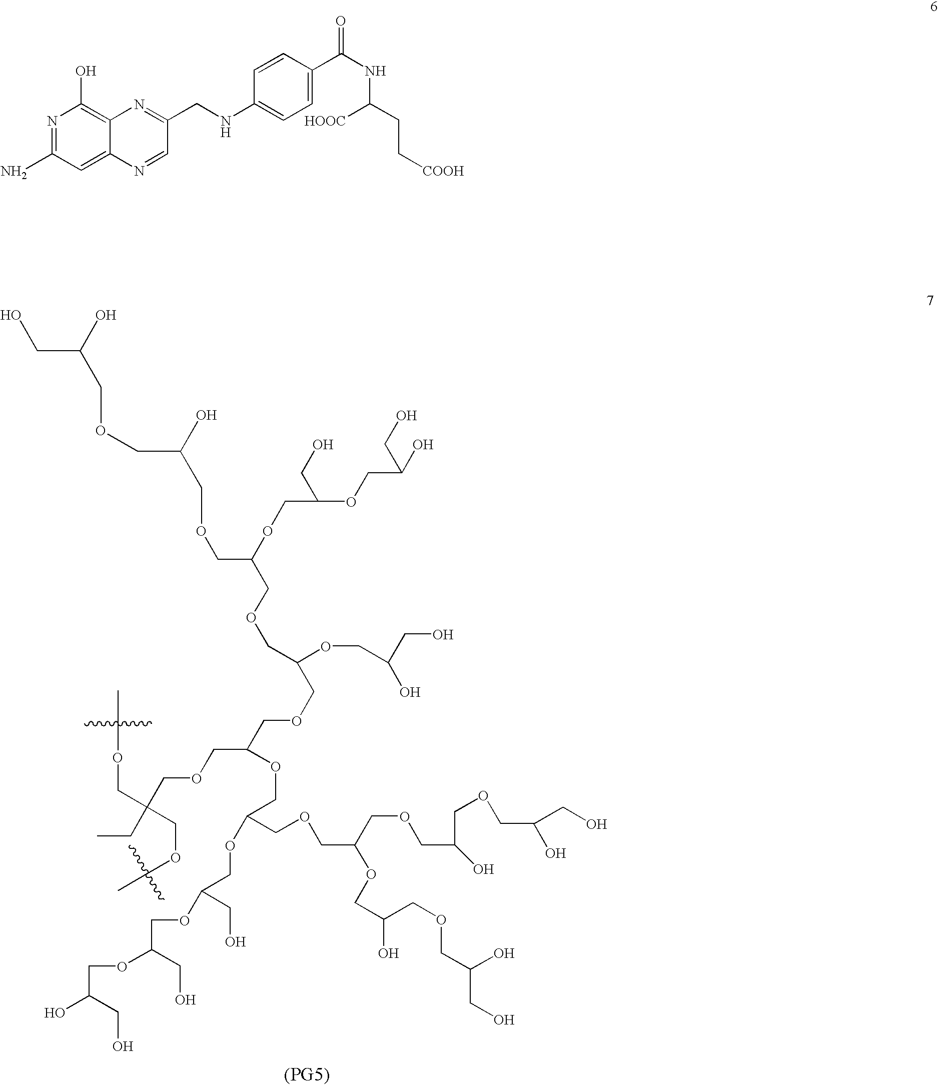
A. Multifunctionalization of Dendrimers

### Example I

Step 1. Diaminobutane poly(propylene imino) dendrimer, 0.001 mol, which is commercially available of the fifth generation (or of any other generation) and 0.004 mol of methoxypoly(ethyleneglycol)-isocyanate of molecular weight 5,000 were dissolved in water. In the resulting solution a small quantity of aqueous triethylamine solution was added for obtaining a solution of pH=13. The solution was stirred for several hours at room temperature. Subsequently the solution was purified by dialysis for 24 hours through a semi-permeable membrane in order that all small molecular weight impurities were removed from the reaction mixture. The introduction of poly(ethylene glycol) moieties in the dendrimer which resulted from Step 1 was established with NMR spectroscopy.

¹H NMR δ=6.20 and 5.90 (s, NHCONH), 3.55 (s, OCH₂CH₂O), 3.25 (s, OCH₃), 3.15 (m, CH₂NHCONHCH₂), 2.70 (m, CH₂NH₂), 2.45 (m, NCH₂CH₂CH₂N, NCH₂CH₂CH₂CH₂N, NCH₂CH₂CH₂NH₂, NCH₂CH₂CH₂NH), 1.55 (m, NCH₂CH₂CH₂N, NCH₂CH₂CH₂CH₂N, NCH₂CH₂CH₂NH), 1.42 (NH₂).

¹³C NMR δ=159.7 (NHCONH), 71.5 (OCH₂CH₂O), 58.5 (OCH₃), 53.5 (NCH₂CH₂CH₂N, NCH₂CH₂CH₂CH₂N), 51.2 (NCH₂CH₂CH₂NH₂), 50.5 (NCH₂CH₂CH₂NHCO), 43.5 (NHCONHCH₂CH₂), 42.4 (NCH₂CH₂CH₂NHCO), 39.5 (CH₂NH₂), 30.4 (CH₂CH₂NH₂), 27.9 (NCH₂CH₂CH₂NHCO), 24.8 (NCH₂CH₂CH₂N, NCH₂CH₂CH₂CH₂N).

Step 2. To 0.001 mol of I dissolved in water, 0.052 mol of ethylisocyanate, dissolved also in water was added. The pH of the solution was adjusted to 13 by adding aqueous 40% trimethylamine solution. The mixture was allowed to react for several hours at room temperature, dialyzed with a 12,400 cut-off membrane for removing low molecular weight compounds and finally lyophilized affording compound II. This second step of functionalization was established by ¹H and ¹³C NMR.

¹H NMR (500 MHz, DMSO-d₆) δ=6.05 (broad s, NHCONH), 3.50 (s, OCH₂CH₂O), 3.25 (s, OCH₃), 3.05 (m, CH₂NHCONHCH₂), 2.70 (m, CH₂NH₂), 2.35 (m, NCH₂CH₂CH₂N, NCH₂CH₂CH₂CH₂N, NCH₂CH₂CH₂NH₂, NCH₂CH₂CH₂NH), 1.45 (m, NCH₂CH₂CH₂N, NCH₂CH₂CH₂CH₂N, NCH₂CH₂CH₂NH), 1.35 (NH₂), 0.98 (t, CH₃).

¹³C NMR (62.9 MHz, D₂O) δ=159.7 (NHCONH), 71.5 (OCH₂CH₂O), 58.5 (OCH₃), 53.5 (NCH₂CH₂CH₂N, NCH₂CH₂CH₂CH₂N), 51.2 (NCH₂CH₂CH₂NH₂). 50.5 (NCH₂CH₂CH₂NHCO), 43.5 (NHCONHCH₂CH₂O), 42.4 (NCH₂CH₂CH₂NHCO), 39.5 (CH₂NH₂), 37.8 (NHCONHCH₂CH₃), 30.4 (CH₂CH₂NH₂), 27.9 (NCH₂CH₂CH₂NHCO), 24.8 (NCH₂CH₂CH₂N, NCH₂CH₂CH₂CH₂N), 14.8 (CH₃).

Step 3. To 0.001 mol of the dendrimer prepared in STEP 1 dissolved in dry DMF, 0.01 mol of 1H-pyrazolo-1-carboxamidine hydrochloride and 0.01 mol of diisopropylethylamine, also dissolved in dry DMF, were added. The reaction mixture was allowed to react overnight at room temperature and the product obtained was precipitated with diethylether and centrifuged. The solid compound was dissolved in water and dialyzed with a 12,400 cut-off membrane. The solvent was removed and the remaining material was extensively dried affording compound III. The introduction of guanidinium group was established by ¹H and ¹³C NMR.

¹H NMR (500 MHz, DMSO-d6) δ=7.65 (broad s, NH of guanidinium group), 6.95 (broad s, NH₂⁺), 6.05 (broad s, NHCONH), 3.50 (s, OCH₂CH₂O), 3.25 (s, OCH₃), 3.05 (m, CH₂NHCONHCH₂, NCH₂CH₂CH₂NHC(NH₂)₂⁺), 2.35 (m, NCH₂CH₂CH₂N, NCH₂CH₂CH₂CH₂N, NCH₂CH₂CH₂NH), 1.45 (m, NCH₂CH₂CH₂N, NCH₂CH₂CH₂CH₂N, NCH₂CH₂CH₂NH), 0.98 (t, CH₃).

¹³C NMR (62.9 MHz, D₂0) δ=159.7 (NHCONH), 157.2 (NHC(NH₂)₂⁺), 71.5 (OCH₂CH₂O), 58.5 (OCH₃), 53.5 (NCH₂CH₂CH₂N, NCH₂CH₂CH₂CH₂N), 50.5 (NCH₂CH₂CH₂NHCO, NCH₂CH₂CH₂NHC(NH₂)₂⁺), 43.5 (NHCONHCH₂CH₂O), 42.4 (NCH₂CH₂CH₂NHCO), 42.2 (NCH₂CH₂CH₂NHC(NH₂)₂⁺), 37.8 (NHCONHCH₂CH₃), 28.2 (NCH₂CH₂CH₂NHC(NH₂)₂⁺), 27.9 (NCH₂CH₂CH₂NHCO), 24.8 (NCH₂CH₂CH₂N, NCH₂CH₂CH₂CH₂N), 14.8 (CH₃

### EXAMPLE II

Step 1. Quaternization of Diaminobutane poly(propyleneimine)dendrimer.

Partial quaternization of poly(propyleneimine) dendrimer was performed as follows: To a solution of 0.113 mmol of DAB-32 (0.398 g) in 10 ml of water, 1.938 mmol of glycidyl trimethylammonium chloride (260 μl) were added. The mixture was allowed to react overnight. It was then dialyzed against H₂O with a 1200 cut-off membrane, for removing unreacted epoxide, and lyophilized. The introduction of the quaternary ammonium was established by ¹H NMR and ¹³C NMR spectra which were recorded in D₂O. The appearance of the expected four new signals at 2.60, 3.16, 3.34 and 4.26 ppm on the ¹H NMR spectrum and at 55.1, 56.9, 67.4 and 71.8 ppm for ¹³C NMR spectrum confirmed that quaternization occurred. Additionally, two new signals appeared at the 13C NMR spectrum at 28.0 and 49.5 ppm, corresponding to the α and β methylene carbons relative to the newly formed secondary amino groups. The degree of substitution was estimated from the integral ratio of the signal at 3.16 ppm, which corresponds to the quaternary methyl protons, relative to the signal at 1.58 ppm, which corresponds to all the β-methylene protons attached to the tertiary, secondary and primary amino groups of the dendrimer. The degree of substitution was found to be 33%.

Synthesis of Folic Acid Active Ester. This is an organic intermediate which is not commercially available and it is required for the next step for preparing the multifunctional dendrimer by the following procedure: Folic acid, 0.594 mmol, dissolved in 7.5 ml of anhydrous DMSO were allowed to react with 0.595 mmol of TEA (82.5 μl) and 0.595 mmol of DCC (0.123 g) in 1 ml of anhydrous solvent for 1 hour under argon atmosphere. 0.594 mmol of N-hydroxy-succinimide in 1 ml of dry DMSO was added to the mixture, which was allowed to react overnight under inert conditions. DCU was removed by filtration and the product was precipitated into dry Et₂O and collected by filtration. The active ester was dried under vacuum for almost 2 hours and was then used for its reaction with the previously obtained quaternized DAB-32.

Step 2 Introduction of folic acid to quaternized DAB-32.

The previously prepared Folic Acid Active Ester is used as a starting material for the introduction of folate targeting ligand to the Dendrimer according to the following procedure: A solution of 0.0137 mmol of quaternized DAB-32 in 7 ml of anhydrous DMSO was added to 0.0413 mmol of folate-NHS active ester dissolved in 1 ml of the same dry solvent. Following a period of 5 days, the product was precipitated into dry Et₂O, dialyzed firstly against phosphate buffer pH 7.4, and afterwards against deionised H₂O with a 1200 cut-off membrane and lyophilized.

Both, ¹H and ¹³C NMR spectra were recorded in D₂O. The presence of the folic acid was confirmed by the characteristic signals at 8.6 ppm, corresponding to the methine group at position 7 of the pterin ring, as well as by the two doublets at 6.7 and 7.7 ppm, corresponding to the aromatic protons of the benzylic moiety. The average number of folate molecules per conjugate was estimated from the integral ratio of the signal at 8.6 ppm, which corresponds to the proton at the 7-position of the pterin ring, to the signal at 4.54 ppm, which corresponds to the methine group bearing the hydroxyl group of the glycidyl reagent, that resulted from the opening of the oxiran ring. The average number of folate residues in the dendrimeric derivative was estimated to be 3. Furthermore, the content of folate in these dendrimers was also determined by UV spectroscopy in PBS (pH 7.4), using extinction coefficient value ε₂₈₀=74620 M⁻¹ cm⁻¹. These results were further confirmed by the ¹³C-NMR spectrum. The final product was quaternized (introduction of cationic charges) and functionalized by the targeting folate ligand while its amino groups (primary, secondary and tertiary) can also be protonated in the biological environment exhibiting thus buffering capacity.

B. Functionalization of Hyperbranched Polymers

PEGylation of the polyglycerol PG-5.

To a solution of 0.04094 mmol of PG-5 in 10 ml of water dissolved in aqueous trimethylamine solution of pH 13, 0.1639 mmol of methoxypoly(ethyleneglycol)-isocyanate dissolved in 10 ml of water were added. The mixture was allowed to react for about 4 days under inert atmosphere, dialyzed with a 12,400 cut-off membrane for removing unreacted polymer and PEG-isocyanate, and finally lyophilized and dried under vacuum, to afford PEGylated PG5.

¹H and ¹³C NMR spectra were recorded in D₂O. The appearance of the signal at 3.32 ppm, which corresponds to the terminal methyl group of the reagent, as well as the signal at 3.25 ppm, which corresponds to the α-CH₂ protons relative to the amide bond (CONHCH₂—), confirmed the introduction of PEG moiety. The formation of the PEGylated hyperbranched polyether polyols was also established by ¹³C NMR spectra. The degree of substitution was estimated from the integral ratio of the signal at 3.24 ppm, which corresponds to the α-CH₂ protons relative to the amide bond (CONHCH₂—), to the signal at 0.82 ppm, which corresponds to the methyl group. of the core moiety. The average number of m-PEG moieties per polymer was 2.

Synthesis of NH2-PEG-folate

NH₂-PEG-Folate was synthesised by reacting polyoxyethylene-bis-amine (Nektar, MW 3400) with an equimolar quantity of folic acid in dry dimethylsulfoxide containing one molar equivalent of dicyclohexylcarbodiimide and pyridine. The reaction mixture was stirred overnight in the dark at room temperature. After the end of the reaction a double volume of water was added, and the insoluble by-product, dicyclohexylurea, was removed by centrifugation. The supernatant was then dialysed against 5 mM NaHCO₃ buffer, pH 9.0 and then against deionized water to remove the unreacted folic acid in the mixture (1,200 cut-off). The trace amount of unreacted polyoxyethylene-bis-amine was then removed by batch-adsorption with cellulose phosphate cation exchange resin prewashed with excess 5 mM phosphate buffer, pH 7.0. The product NH₂-PEG-Folate was dialysed once again against water, lyophilized and its ¹H and ¹³C NMR spectra were recorded in D₂O. The presence of the folic acid was confirmed by the characteristic signals in the products ¹H NMR spectrum at 8.64 ppm, corresponding to the methine group at position 7 of the pterin ring, as well as by the two doublets at 6.74 and 7.60 ppm, corresponding to the aromatic protons of the benzylic moiety. The average number of folate molecules per conjugate was estimated from the integral ratio of the signal at 8.64 ppm, to the signal at 3.15 ppm, which corresponds to the α-methylene group next to the remaining amino group. Only the γ-carboxyl group of the folic acid reacted, according to the replacement of the signal of its α-methylene from the 30.4 ppm, by a new peak at 32.6 ppm in the ¹³C NMR spectrum.

Synthesis of PG5-PEG-folate

PG5-PEG-folate was synthesised by reacting overnight in slightly elevated temperature, the polyglycerol PG-5, with an excess of succinic anhydride in DMF, so as to achieve the reaction of a 5-10% of the polyglycerols hydroxyl groups. The product of the reaction was dialysed against water and its structure was confirmed by ¹H and ¹³C NMR experiments. Two new signals appeared at the ¹H NMR spectrum corresponding to the α- and β-methylenes to the newly formed ester bond, at 2.5 and 2.6 ppm, respectively. Additionally, the formation of the amide bond was achieved by reacting NH₂-PEG-folate with the modified polyglycerol PG5 in dry DMF and in the presence of dicyclohexylcarbodiimide and pyridine, as described above. The product of the reaction was dialysed against water (5,000 cut-off), and once again the introduction of the folate was confirmed by ¹H and ¹³C NMR experiments. The presence of the PEG-folate on the hyperbranched polymer was confirmed by the characteristic signals in the ¹H NMR spectrum at 8.64 ppm. The average number of folate molecules per conjugate was estimated from the integral ratio of the signal at 8.64 ppm, to the signal at 0.82 ppm, which corresponds to the methyl group of the polymer core group. Furthermore, the content of folate in our molecules was also determined by quantitative UV spectroscopy of the conjugates in PBS (pH 7.4), using extinction coefficient values ε₂₈₀=74620 M⁻¹ cm⁻¹.

Encapsulation and Release of Betamethasone Derivatives

The encapsulation of betamethasone derivatives in the multifunctional dendrimer prepared in the EXAMPLE 1 was performed with the following method: The dendrimer and the betamethasone valerate derivative were dissolved in a mixture of chloroform/ethanol. A thin film was obtained, after the distillation of the solvent, which was dispersed in water. The dendrimer with the encapsulated compound was taken in the aqueous phase while the non-encapsulated substance remained insoluble in water and was removed with centrifugation. The percentage of the encapsulated Betamethasone Valerate within the multifunctional dendrimer are given in Table 1. For comparison the data from the encapsulation of pyrene, e.g. of a well-known probe are included.

TABLE 1
Comparative solubility of pyrene (PY) and betamethasone valerate (BV) in
Parent and Multi-functional Dendrimer.

| | | | PY/Dendrimer | | BV/Dendrimer |
|---|---|---|---|---|---|
| Compound | [dendrimer]/M | [PY]/M | molar ratio | [BV]/M | molar ratio |
| DAB-64 | 1.0 × 10⁻³ | 2.1_{±0.2} × 10⁻⁵ | 0.021_{±0.002} | 2.5_{±0.4} × 10⁻⁴ | 0.25_{±0.04} |
| Multi-functional | 2.5 × 10⁻⁴ | 1.9_{±0.08} × 10⁻⁵ | 0.076_{±0.002} | 1.80± × 10⁻³ | 7.20_{±0.03} |
| Dendrimer | | | | | |

The release, for example, of Betamethasone Valerate was achieved with gradual addition of sodium chloride aqueous solution (FIG. 6). It is observed that the bioactive compound has been released almost completely from the multi-functional dendrimer upon addition of 0.8 M NaCl.
Preparation of Multi-Functional Dendrimer Carrying Genetic Material

Positively charged multi-functional dendrimer was added to a plasmid DNA (3-7 mg) so that the charge ratio of the dendrimer to DNA to be between 3.5:1 to 8.5:1 in various media such as natural serum, aqueous sodium chloride solution 300 mM, RPMI-1640.

### DETAILED DESCRIPTION OF FIGURES

FIG. 1 shows a molecule of a general formula I with a symmetric dendrimeric structure which is an object of the present invention, where the symbol (●) can be an atom of a chemical element able to form three or more chemical bonds, as for instance nitrogen or an appropriate characteristic group, the straight line (-) corresponds to an aliphatic chain and the external functional groups X, Y, Z are groups that collectively: a) render the molecules of the above polymers recognizable from the complementary receptors of the cells, b) render the same polymers stable in biological environment and c) facilitate the transport of these polymers through cell membranes.

FIGS. 2 and 3 show structures of the molecule of two different non-symmetric hyperbranched polymers, which are objects of the present invention where the symbol (●) can be an atom of a chemical element able to form three or more chemical bonds, as for instance nitrogen or appropriate characteristic group, the straight line (-) corresponds to an aliphatic chain and the external functional groups X, Y, Z are groups that collectively: a) render the molecules of the above polymers recognizable from the complementary receptors of the cells, b) provide to these polymers stability in biological environment and c) they facilitate the transport of these polymers through cell membranes.

FIG. 4 shows the stepwise introduction of functional groups on the surface of a dendrimer (or hyperbranched polymers ) according to one embodiment of the present invention and namely that:

In a first stage there is a reaction of the external amino- or hydroxy-groups of the dendrimer with appropriate polymers bearing reactive groups, as for example, epoxy- or N-hydroxysuccinimide.

In a second stage follows a reaction of the greater part of the amino groups remaining on the dendrimer surface, for example, with ethyl isocyanate for the replacement of the toxic amino group.

In a third stage took place the introduction of recognizing groups, as for example the guanidinium group.

In a fourth stage groups were introduced that facilitate the transfer of the carriers with the encapsulated pharmaceutical compound through the cell membranes, as guanidinium group, oligo-argine or poly-arginine.

FIG. 5 shows schematically the formation of the complex between the dendrimeric carrier and DNA or oligonucleotide and its transport through cell membrane.

FIG. 6 shows the diagram of the release of the encapsulated Betamethasone Valerate as a function of the concentration of aqueous sodium chloride solution.

FIG. 7 shows the introduction of functional groups on the surface of a hyperbranched polymer according to one embodiment of the present invention and namely that in one step reaction two functional groups, e.g. the protective PEG chains and the folate targeting ligand, attached at the terminal OH groups, are introduced.

When used in the specification and claims, the terms “comprise”, “comprising” and variations thereof mean that the specified features, steps, components or integers are included. The terms are not to be interpreted to exclude the presence of other features, steps, components or integers.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination or such features, be utilised for realising the invention in diverse forms thereof.

1. A dendrimeric polymer with symmetric chemical structure or non-symmetric hyperbranched polymer, characterized in that the polymer is modified so as to comprise:
at least one atom of a chemical element able to form three or more chemical bonds
various different terminal functional groups bonded to said at least one atom, which terminal functional groups, together:
a) have low toxicity or no toxicity at all,
b) render the molecules of the above polymers recognizable from the complementary receptors of the cells,
c) render the polymers stable in a biological environment and
d) facilitate the transport of the said polymers through cell membranes.

2. A dendrimeric polymer or hyperbranched non-symmetric polymer according to the claim 1, which polymer is cationized for the formation of complexes with DNA when the said compounds are destined to be carriers of genetic material.

3. A dendrimeric polymer or hyperbranched non-symmetric polymer according to the claim 2, wherein the polymer is cationized by introducing ammonium, quaternary ammonium or guanidinium groups at the terminal groups of the dendrimer.

4. A dendrimeric polymer or hyperbranched non-symmetric polymer according to the claim 1 where the atom of a chemical element able to form three or more chemical bonds, is nitrogen, carbon or silicon.

5. A modified dendrimeric polymer according to the claim 1 which is a modified diaminobutane poly(propylene imino) dendrimer (DAB) or PAMAM dendrimer.

6. A modified hyperbranched non-symmetric polymer according to the claim 1, wherein the hyperbranched polymer is derived from the poly-condensation of an anhydride with a dialkyl amine.

7. A modified hyperbranched non-symmetric polymer according to the claim 1, wherein the hyperbranched polymer is derived from the anionic polymerization of epoxide derivatives with 1,1,1 tri(hydroxyalkyl)propane.

8. A modified hyperbranched non-symmetric polymer according to the claim 1, wherein the hyperbranched polymer is derived from the anionic polymerization of glycidol with 1,1,1 tri(hydroxymethyl)propane (PG-5).

9. A modified dendrimeric polymer or modified hyperbranched non-symmetric to polymer according to claim 1, wherein the functional groups include polymeric chains of diversified molecular weight at the surface of the dendrimeric polymeric or of the hyperbranched polymer.

10. A modified dendrimeric polymer or modified hyperbranched non-symmetric polymer according to claim 1, wherein the functional groups include at least one group that is complementary to a receptor site of a cell, a carbohydrate a folate, an RGD receptor, a nucleobase moiety or a barbiturate.

11. A modified dendrimeric polymer or modified hyperbranched non-symmetric polymer according to claim 1, wherein the functional groups include at least one group that facilitates the transport of the dendrimeric polymer or modified hyperbranched polymer together with any encapsulated active drug ingredient or genetic material through a cell membrane, e.g. a guanidinium as moiety, an oligoarginine or polyarginine derivative or a polypropylene oxide moiety.

12. A modified dendrimeric polymer or modified hyperbranched non-symmetric polymer according to claim 1, wherein the functional groups include at least one targeting ligand, a carbohydrate, a folate, an RGD receptor, a nucleobase moiety or a barbiturate.

13. A dendrimeric polymer or hyperbranched non-symmetric polymers according to claim 1, including an encapsulated bio-active pharmaceutical compound or carrying genetic material.

14. A dendrimeric polymer or hyperbranched non-symmetric polymer according to claim 1, wherein the bio-active pharmaceutical compound is betamethasone or a betamethasone derivative.

15. A method for the synthesis of a multi functional dendrimer or hyperbranched polymer according to claim 1 which method is characterized in that the surface of these polymers is modified in steps that comprise:
a) Substitution of the amino groups or other toxic groups of the surface with hydroxy, carboxylic or quaternary ammonium groups or other non-toxic groups
b) Introduction of polymeric chains of diversified molecular weight at the surface of the dendrimeric carriers or of the hyperbranched polymers, as for instance of poly(ethyleneglycol) (PEGylation) so that the polymers are thus protected from the MPS (Mononuclear Phagocyte System) of the organism.
c) Introduction of recognizable groups complementary to the receptors or to the tissues, carbohydrate moieties, folate or RGD receptor, nucleobase moieties or barbiturate group, so as to enhance the targeting ability of the carrier, and
d) Introduction of groups that facilitate the transport of the carriers together with the encapsulated bio-active pharmaceutical compound through cell membranes, such as guanidinium moieties, oligo-arginine or poly-arginine derivatives or polypropylene oxide moieties.

16. A method according to claim 15 where
the initial reaction of external amino or hydroxy groups of dendrimers or hyperbranched polymers is performed with appropriate protective polymers, bearing reactive groups at one end such as isocyanate, epoxide or N-hydroxysuccinimide,
subsequent reaction of the greatest portion of amino groups of the obtained polymer is performed with ethylisocyanate for the replacement of toxic amino groups,
subsequent-reaction of the previously obtained polymer for the transformation of amino groups to recognizable groups as for example guanidinium groups,
subsequent introduction of a group or groups which facilitate the transport of the carriers through cell membranes as for instance polyarginine or propyleneoxide chains.

17. A method according to claim 15, which method is characterized in that the said polymers are cationized for the formation of complexes with DNA.

18. A method according to claim 15, which method is characterized in that when the toxic group of the surface is an amino group, a aliphatic chain having less than eight carbon atoms, preferably two or three carbon atoms, is introduced for its replacement.

19. A pharmaceutical formulation characterized in that it comprises a bio-active pharmaceutical compound or genetic material encapsulated in a modified multi-functional dendrimeric or modified multi-functional hyperbranched non-symmetric polymer according to claim 1.

20. A method for producing a pharmaceutical formulation for delivering a bio-active pharmaceutical compound or genetic material, which method comprises synthesizing a polymer according to claim 15 and encapsulating the bio-active pharmaceutical compound or genetic material therewith.

21. A modified dendrimeric polymer or a modified hyperbranched non-symmetric polymer according to claim 1 that includes an encapsulated bio-active pharmaceutical compound or that carries genetic material for use in therapy.

22. Use of a modified dendrimeric polymer or a modified hyperbranched non-symmetric polymer according to claim 1 that includes an encapsulated bio-active pharmaceutical compound or that carries genetic material in therapy, for manufacture of a pharmaceutical dosage form.

23. Use of a modified dendrimeric polymer or a modified hyperbranched non-symmetric polymer according to claim 1 that includes an encapsulated bio-active pharmaceutical compound or that carry genetic material in the manufacture of a medicament for treating the same disease or condition as the compound or the genetic material.

24. A modified hyperbranched non-symmetric polymer according to the claim 6 wherein the anhydride is succinic anhydride, phthallic anhydride or tetrahydrophthalic anhydride and the dialkyl amine is diisopropylamine.

25. A modified hyperbranched non-symmetric polymer according to the claim 9 wherein the polymeric chains are polyalkyl glycol or poly(ethyleneglycol).

26. A modified hyperbranched non-symmetric polymer according to the claim 10 wherein the cell is a guanidinium group, the carbohydrate is mannose, glycose, or galactose, and the nucleobase moiety is adenine, thymine, guanine, or cytosine.

27. A modified hyperbranched non-symmetric polymer according to the claim 12 wherein the targeting ligand is guanidinium group, the carbohydrate is mannose, glycose, or galactose, and the nucleobase moiety is adenine, thymine, guanine, or cytosine.
